(19) United States
(12) Patent Application Publication
(10) Pub. No.: US 2007/0021336 A1
(43) Pub. Date: Jan. 25, 2007

(54) Use of GLP-1 and agonists thereof to prevent cardiac myocyte apoptosis

(76) Inventors: Christen Anderson, Encinitas, CA (US); Alain D. Baron, San Diego, CA (US)

Correspondence Address: ARNOLD & PORTER LLP (18528);ATTN; IP DOCKETING DEPT., 555 TWELFTH ST, NW, WASHINGTON, DC, 20004, US

(21) Appl. No.: 11/313,763

(22) Filed: Dec. 22, 2005

Related U.S. Application Data
(60) Provisional application No. 60/639,124, filed on Dec. 24, 2004.

(51) Int. Cl. A61K 38/26

(52) U.S. Cl. 514/12

(57) ABSTRACT

The present invention relates generally to the novel use of GLP-1, including analogs, and agonists, to prevent cardiac myocyte apoptosis. The present invention relates to methods for using GLP-1 for the treatment of conditions associated with cardiac myocyte apoptosis. The present invention further relates to improving the efficiency of cardiac myocytes and also to improving cardiac contractility.

### CROSS REFERENCE TO RELATED APPLICATIONS

This application claims the benefit of United States Provisional Application Ser. No. 60/639,124, filed Dec. 24, 2004, which is herein incorporated by reference in its entirety for all purposes.

### FIELD OF THE INVENTION

The present invention relates generally to the use of GLP-1 molecules or agonists thereof, and more particularly to the use of GLP-1 molecules or agonists thereof in treatment or prevention of various cardiac diseases or disorders.

### BACKGROUND OF THE INVENTION

The contractile cells of the heart are referred to as cardiac myocytes. Myocytes are terminally differentiated cells that are generally withdrawn from the cell cycle during the perinatal period. As such, death of myocytes has a significant negative impact on cardiac function. Although in the short term following death of some myocytes, surviving myocytes may undergo a compensatory hypertrophic growth response to maintain cardiac output, this response is not sustained and heart failure may result.

Congestive heart failure is one of the most significant causes of morbidity and mortality in developed countries. It occurs as a late manifestation in diverse cardiovascular diseases characterized by loss of contractile mass and/or by volume or pressure overload (Fortuno, Hypertension 38: 1406-1412 (2001)). Numerous studies have proposed that myocyte loss in cardiomyopathy can occur by apoptosis (Okafor, BMC Physiology 3:6 (2003)).

Apoptosis is an energy-requiring physiological mechanism of cell deletion. Apoptosis is a predominant and ubiquitous physiological mode of cell death distinct from cell mortality caused by necrosis. Apoptosis is often referred to as programmed cell death because it is a genetically directed process that occurs in response to internal or external stimuli. Apoptosis is readily distinguishable from necrotic mechanisms because unlike the latter, the former typically produces DNA fragmentation and laddering and ultimately morphological changes. In addition, whereas swelling and rupture are generally associated with necrosis, apoptotic cells generally shrink, maintain membrane integrity, and are cleared by neighboring cells or macrophages.

It has been reported that cardiac myocyte apoptosis can occur in response to conditions such as, for example, heart failure (See e.g., Narula, New Eng. J. Med. 335(16): 1182-1189 (1996); Olivetti, New Eng. J. Med. 336(16): 1131-1141 (1997)), myocardial infarction (See e.g., Olivetti, J. Mol. Cell. Cardiol. 28: 2005-2016 (1996)), ischemia/reperfusion (See e.g., MacLellan, Circulation Research 81:137-144 (1997)), oxidative stress (See e.g., Singh, J. Cell. Physiol. 189: 257-265 (2001)), advanced glycation endproducts (as in diabetes, Fiordaliso, Diabetes 50: 2363-2375 (2001)), abnormal cardiac wall tension (as in some forms of heart failure, Jiang, European Heart Journal 24: 742-751 (2003)), sympathetic stimulation (Singh, J. Cell. Physiol. 189: 257-265 (2001)), myocarditis (See id.), hypertension (Fortuno, Hypertension 38:1406-1412 (2001)), and heart transplantation (Miller, Cardiovascular Disease 19(1): 141-154 (2001)). In each case, loss of myocardium through apoptosis is believed to contribute to a decline in cardiac function. As such, agents that act to prevent or decrease apoptosis of cardiac myocytes are desired. Indeed, the literature has identified a need for molecules that can blunt the mechanisms of cardiac myocyte apoptosis (Fortuno, Hypertension 38:1406-1412 (2001)).

Literature reports indicate that GLP- 1 released from gut endocrine L cells is a regulator of apoptosis in pancreatic β-cells (Drucker, Molecular Endocrinology 17(2):161-171 (2003)). More particularly, GLP-1 has been used to ameliorate the age-related decline in pancreatic β-cell function by increasing both the number of cells secreting insulin as well as the amount of insulin secreted per cell (See e.g., Doyle, Recent Progress in Hormone Research 56(1): 377-400 (2001)). According to the literature, GLP-1 released from the pancreas acts by activating a GLP-1 receptor, which receptor has been identified as a 463-amino acid member of the G protein-coupled receptor superfamily (Drucker, Diabetes 47: 159-169 (1998)). It has been reported that the GLP-1 receptor in cardiac myocytes is structurally identical to the pancreatic islet receptor (See id.).

While there are many treatments available for congestive heart failure, only one agent has been shown to actually decrease the loss of cardiac myocytes (i.e., carvedilol). All of the other agents improve cardiac function by blocking neurohormonal stimulation (e.g., beta adrenergic blockers, aldosterone antagonist), by increasing neurohormal stimulation (e.g., brain natriuretic peptide, dobutamine infusion), or by indirectly altering preload or afterload (e.g., angiotensin convering enzyme inhibition, angiotensin receptor antagonists, diuretics). Carvedilol is a β-adrenergic blocking drug that has been reported to decrease the incidence of apoptosis in cardiac myocytes (Okafor, BMC Physiology 3:6 (2003)). Carvedilol activities include nonselective blockade of β-adrenoceptors, vasodilation and antioxidant activity. Despite the ongoing research and development of treatments for congestive heart failure, there is till a tremendous need for improved and alternative treatments.

### SUMMARY OF THE INVENTION

The present invention relates generally to the use of GLP-1 molecules or agonists thereof to prevent cardiac myocyte apoptosis. In one aspect, the present invention relates to methods for using GLP-1 for the treatment of conditions associated with cardiac myocyte apoptosis. In another aspect, the present invention further relates to improving the efficiency of cardiac myocytes and also to improving cardiac contractility.

In one embodiment, a method for preventing or ameliorating apoptosis of cardiac myocytes in a subject in need thereof is provided. The method comprises administering to the subject an amount of a GLP-1 molecule or agonist thereof effective to prevent cardiac myocyte apoptosis.

In another embodiment, a method for improving cardiac contractility in a subject in need thereof is provided. The method generally comprises administering to the subject an amount of a GLP-1 molecule or agonist thereof effective to improve cardiac contractility in the subject.

In yet another embodiment, a method for improving the efficiency of cardiac myocytes in a subject in need thereof is provided. The method generally comprises administering to the subject an amount of a GLP-1 molecule or agonist thereof effective to improve efficiency of cardiac myocytes in the subject.

In yet another embodiment, a method for the treatment or prevention of a condition associated with cardiac myocyte apoptosis in a subject in need thereof is provided. The method generally comprises administering to the subject an amount of a GLP-1 molecule or agonist thereof effective to prevent or ameliorate apoptosis of cardiac myocytes, wherein the condition associated with cardiac myocyte apoptosis is thereby improved.

### BRIEF DESCRIPTION OF THE DRAWINGS

FIGS. 1A and 1B illustrate certain preferred exendin compounds of the invention.

### DETAILED DESCRIPTION OF THE INVENTION

The present invention generally provides methods for preventing or ameliorating apoptosis of cardiac myocytes. In general, apoptosis refers to a form or mechanism of cell death. As described above and without intending to be limited by theory, apoptosis is often described as programmed cell death because it is generally thought to constitute a genetically directed process that occurs in response to internal or external stimuli. As such, apoptosis can be described as an energy-requiring physiological mechanism of cell deletion. Apoptosis often can be distinguished from necrotic mechanisms because unlike necrosis, apoptosis typically produces DNA fragmentation and laddering and ultimately morphological changes, such as the formation of membrane blebs and apoptotic bodies, chromatin and nuclear condensation, and the dismantling of organelles. In addition, whereas swelling and rupture are generally associated with necrosis, apoptotic cells generally shrink, maintain membrane integrity, and are cleared by neighboring cells or macrophages.

The apoptosis of cardiac myocytes can include apoptosis that occurs in response to any stimulus or combination of stimuli. By way of non-limiting example, apoptosis of cardiac myocytes can occur in response to cardiac surgery, heart failure, myocardial infarction, ischemia/reperfusion, oxidative stress, cardioplegia, advanced glycation endproducts (as occurs in diabetes), abnormal cardiac wall tension (as occurs in some forms of heart failure), sympathetic stimulation, myocarditis, hypertension, and heart transplantation.

A. Methods of the Invention

In an aspect of the present invention, apoptosis of cardiac myocytes is prevented or ameliorated by the administration of a GLP-1 molecule or agonist thereof. In the context of the present invention, prevention or amelioration of apoptosis can include a reduction of apoptosis by any amount. In one embodiment, prevention or amelioration of apoptosis is accompanied by an improvement in myocyte efficiency.

In an embodiment, apoptosis is ameliorated or reduced to an amount that is less than about 1%, 2%, 5%, 10%, 20%, 30%, 40%, 50%, 60%, 70%, 80%, or 90% of the amount of apoptosis in the absence of a GLP-1 molecule or agonist thereof administration. In another embodiment, apoptosis can be slightly reduced, moderately reduced, or substantially eliminated, as compared to the occurrence of apoptosis in the absence of administering a GLP-1 molecule or agonist thereof. As used herein, a slight reduction of apoptosis refers to apoptosis that is decreased by about 25% or less as compared with apoptosis in the absence of administering a GLP-1 molecule or agonist thereof. A moderate reduction in apoptosis refers to apoptosis that decreased by about 50% or less as compared with apoptosis in the absence of administering a GLP-1 molecule or agonist thereof. A substantial elimination of apoptosis refers to apoptosis that is decreased by about 90% or more as compared with apoptosis in the absence of administering a GLP-1 molecule or agonist thereof.

In order to assess the degree to which apoptosis is prevented, any means available to the skilled art worker can be employed. For example, apoptosis can be assessed by analyses including but not limited to DNA laddering, terminal deoxynucleotidyl transferase (TdT)-mediated nick end-labeling (TUNEL) assay, and flow cytometric analysis of cellular DNA content.

DNA laddering can be assessed by any means available in the art, for example, by performing agarose gel electrophoresis of genomic DNA molecules. Apoptosis tends to be characterized by degradation of chromosomal DNA into fragments that are multiples of 180 base pairs. In one aspect of the present invention, such fragments can be labeled with radionucleotides, resolved on an agarose gel containing ethidium bromide, and subjected to autoradiography.

Alternatively, a TUNEL assay can be performed on cardiac myocytes in any manner available to the artisan, such as, for example, by using a death detection kit according to manufacturer's instructions (see e.g., In situ Cell Death Detection Kit, Roche Applied Science, Indianapolis, Ind.). The percentage of myocytes exhibiting DNA that is nick end-labeled can be quantified, for example, by counting cells that possess fluorescent green nuclei.

Flow cytometry can be used to assess apoptosis. The skilled artisan can use any desired parameters to conduct flow cytometry studies. In a preferred embodiment, cells are stained with propidium iodide, and a FACScan is used with excitation at 488 nm and emission measured at 560 nm to 640 nm. In a preferred embodiment, apoptotic cells exhibit reduced DNA content and a peak in the hypodiploid region. Methods for analyzing cells by flow cytometry are well known in the art and can be found, for example, in Watson, Introduction to Flow Cytometry, Cambridge Univ. Press, 2004; Shapiro, Practical Flow Cytometry, 4^{th} ed., Wiley-Liss, 2003; Steensam et al., Methods Molec. Med. 85:323-332, 2003; Vernes et al., J. Immunol. Methods 243:167-190, 2000; and Ormerod, Leukemia 12:1013-1025, 1998.

In an embodiment, the methods of the present invention contemplate administering to a sample or subject an amount of one or more GLP-1 molecules or agonists thereof effective to prevent cardiac myocyte apoptosis. A sample includes any material that contains one or more cardiac myocytes. For example, a sample can include one or more cells, tissues, or cultures. An exemplary sample is a human heart. A subject can be any organism that comprises one or more cardiac myocyte cells. The cardiac myocyte cells can be native to the organism, or alternatively, the cardiac myocytes can be introduced, such as for example by transplantation. Exemplary non-limiting subjects include organisms such as pigs, mice, rats, dogs, cats, chickens, sheep, goats, cattle, and humans. In one embodiment the subject is a human.

In an embodiment of the present invention, samples and subjects that may be benefited by administration of a GLP-1 molecule or agonist thereof to prevent cardiac myocyte apoptosis can be ascertained by the artisan in light of conditions and risk factors related to the sample or subject. Samples and subjects of the present invention include those which have experienced, are experiencing or are at risk to experience a condition associated with cardiac myocyte apoptosis. A condition associated with cardiac myocyte apoptosis can be any condition or disorder in which myocyte apoptosis is known to occur or thought to be a risk. Conditions associated with cardiac myocyte apoptosis include, for example, myocardial infarction, ischemia/reperfusion, oxidative stress, advanced glycation endproducts, abnormal cardiac wall tension, sympathetic stimulation, myocarditis, hypertension, and heart transplantation.

In accordance with the methods of the present invention, the GLP-1 molecules or agonists thereof may be administered in any manner known in the art that renders a GLP-1 molecule or agonist thereof biologically available to the subject or sample in an effective amount. For example, the GLP-1 molecule or agonist thereof may be administered to a subject via any central or peripheral route known in the art including, but not limited to: oral, parenteral, transdermal, transmucosal, or pulmonary routes. Particularly preferred is parenteral administration. Exemplary routes of administration include oral, ocular, rectal, buccal, topical, nasal, ophthalmic, subcutaneous, intramuscular, intraveneous, intracerebral, transdermal, and pulmonary. In one embodiment, the route of administration is subcutaneous. Further, the GLP-1 molecules or agonists thereof can be administered to a sample via pouring, pipetting, immersing, injecting, infusing, perfusing, or any other means known in the art. Determination of the appropriate administration method is usually made upon consideration of the condition (e.g., disease or disorder) to be treated, the stage of the condition (e.g., disease or disorder), the comfort of the subject, and other factors known to those of skill in the art.

Administration by the methods of the present invention can be intermittent or continuous, both on an acute and/or chronic basis. One method of administration of a GLP-1 molecule or agonist thereof is continuous. Continuous intravenous or subcutaneous infusion, and continuous transcutaneous infusion are exemplary embodiments of administration for use in the methods of the present invention. Subcutaneous infusions, both acute and chronic, are other embodiments of administration.

In one embodiment, administration of a GLP-1 molecule or agonist thereof to prevent cardiac myocyte apoptosis can be a prophylactic treatment, beginning concurrently with the diagnosis of conditions (e.g., disease or disorder) which places a subject at risk of cardiac myocyte apoptosis, such as for example upon a diagnosis of diabetes. In the alternative, administration of a GLP-1 molecule or agonist thereof to prevent cardiac myocyte apoptosis can occur subsequent to occurrence of symptoms associated with cardiac myocyte apoptosis.

The term “effective amount” refers to an amount of a pharmaceutical agent used to treat, ameliorate, prevent, or eliminate the identified condition (e.g., disease or disorder), or to exhibit a detectable therapeutic or preventative effect. The effect can be detected by, for example, chemical markers, antigen levels, or time to a measurable event, such as morbidity or mortality. Therapeutic effects include preventing further loss of cardiac myocytes, or improving cardiac myocyte efficiency, or both. Therapeutic effects also include an improvement in cardiac contractility. Further therapeutic effects include reduction in physical symptoms of a subject, such as, for example, an increased capacity for physical activity prior to breathlessness. The precise effective amount for a subject will depend upon the subject's body weight, size, and health; the nature and extent of the condition; and the therapeutic or combination of therapeutics selected for administration. Effective amounts for a given situation can be determined by routine experimentation that is within the skill and judgment of the clinician.

For any GLP-1 molecule or agonist thereof, the effective amount can be estimated initially either in cell culture assays, e.g., in animal models, such as rat or mouse models. An animal model may also be used to determine the appropriate concentration range and route of administration. Such information can then be used to determine useful doses and routes for administration in humans.

Efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between therapeutic and toxic effects is the therapeutic index, and it can be expressed as the ratio, ED₅₀/LD₅₀. Pharmaceutical compositions that exhibit large therapeutic indices are preferred. The data obtained from cell culture assays and animal studies may be used in formulating a range of dosage for human use. The dosage contained in such compositions is preferably within a range of circulating concentrations that include an ED₅₀ with little or no toxicity. The dosage may vary within this range depending upon the dosage form employed, sensitivity of the patient, and the route of administration.

More specifically, the concentration-biological effect relationships observed with regard to the GLP-1 molecules or agonists thereof employed in the methods of the present invention indicate an initial target plasma concentration ranging from about 5 pM to about 400 pM, preferably from about 20 pM to about 200 pM, more preferably from about 80 pM to about 100 pM. To achieve such plasma concentrations in the methods of the present invention, a GLP-1 molecule or agonist thereof may be administered at doses that vary from about 0.25 pmol/kg/min to about 10 nmol/kg/min, more preferably about 0.45 pmol/kg/min to about 4.5 nmol/kg/min, depending upon the route of administration. Guidance as to particular dosages and methods of delivery is generally available to practitioners in the art and is provided herein.

In general, for continuous subcutaneous infusion, the dose will be in the range of about 0.2 pmol/kg/min to about 13 pmol/kg/min, or from about 0.3 pmol/kg/min to about 11 pmol/kg/min, or from about 0.45 pmol/kg/min to about 8.5 pmol/kg/min. For acute subcutaneous infusion, the dose will generally be in the range of about 2.5 pmol/kg/min to about 7 nmol/kg/min, or from about 3.5 pmol/kg/min to about 6 pmol/kg/min, or from about 5 pmol/kg/min to about 4.5 nmol/kg/min. The exact dosage will be determined by the practitioner, in light of factors related to the subject that requires treatment.

As mentioned above, the GLP-1 molecule or agonist thereof may be administered on an acute or chronic basis. An acute administration includes a temporary administration for a period of time before, during and/or after the occurrence of a transient event. An acute administration generally entails an administration that is indicated by a transient event or condition. For example, acute administration may be implicated during an evolving myocardial infarction or during unstable angina. Administration before, during, and/or after a percutaneous cardiac intervention (“PCI”) also constitutes an example of an acute administration. In addition, GLP-1 molecules or agonists thereof may be administered acutely before, during and/or after any cardiac surgery, such as open heart surgery, coronary bypass, minimally invasive cardiac surgery, valvuloplasty, or cardiac transplantation. Alternatively, GLP-1 may also be administered acutely on the basis of congestive heart failure following myocardial infarction or surgery.

Acute administration before, during, and/or after a particular event may begin at any time before the happening of the event (e.g., such as surgery or transplant) and may continue for any length of time, including for an extended period of time after the event, that is useful to prevent or ameliorate cardiac myocyte apoptosis associated with the event. The duration of an acute administration can be determined by a clinician in light of the risk of cardiac myocyte apoptosis related to the event or condition.

Chronic administration of a GLP-1 molecule or agonist thereof for the prevention or amelioration of apoptosis in cardiac myocytes may be warranted where no particular transient event or transient condition associated with apoptosis is identified. Chronic administration includes administration of a GLP-1 molecule or agonist thereof for an indefinite period of time on the basis of a general predisposition to cardiac myocyte apoptosis or on the basis of a predisposing condition that is non-transient (e.g., a condition that is non-transient may be unidentified or unamenable to elimination, such as diabetes). A GLP-1 molecule or agonist thereof may be administered chronically in the methods of the invention in order to prevent cardiac myocyte apoptosis in a subject who exhibits congestive heart failure, regardless of etiology. Chronic administration of a GLP-1 molecule or agonist thereof for the prevention or amelioration of cardiac myocyte apoptosis may also be implicated in diabetics at risk for congestive heart failure. GLP-1 may also be administered on a chronic basis in order to preserve a transplanted organ in individuals who have received a heart transplant. When a GLP-1 molecule or agonist thereof is administered chronically, administration may continue for any length of time. However, chronic administration often occurs for an extended period of time. For example, in one embodiment, chronic administration continues for 6 months, 1 year, 2 years or longer.

In another embodiment, the methods of the present invention also include administration of a GLP-1 molecule or agonist thereof to improve cardiac contractility. Improving cardiac contractility may include any increase in the number of cardiac myocytes available for contraction, the ability of cardiac myocytes to contract, or both. In order to evaluate the improvement of cardiac contractility, any mode of assessment may be used. For example, clinical observation, such as an increase in cardiac output or a decrease in cardiac rate or both, may lead to a determination of increased cardiac contractility. Alternatively, in vivo an increased contractility of the heart may be assessed by a determination of an increased fractional shortening of the left ventricle. Fractional shortening of the left ventricle may be observed by any available means such as echocardiograph.

In evaluating increased cardiac contractility, the increase in fractional shortening of the left ventricle may be an increase of any amount as compared with the fractional shortening before administration of a GLP-1 molecule or agonist thereof. For example, the increase in shortening may be about 10%, 20%, 30%, 40%, 50%, 60%, 70%, 80%, 90%, 100%, 150%, 200% or more than about 200%.

In yet another aspect of the present invention, a method for improving the efficiency of cardiac myocytes by the administration of a GLP-1 molecule or agonist thereof is provided. Improving the efficiency of cardiac myocytes may be evaluated as compared to efficiency of cardiac myocytes before administration of a GLP-1 molecule or agonist thereof, and may include any increase in the work done by a cardiac myocyte or any decrease in the time required for a cardiac myocyte to act. Improved efficiency of cardiac myocytes may be evaluated by any means available to the skilled artisan.

By way of example, assessment of improved myocyte efficiency may be conducted by observing increased contractility of cardiac myocytes as described previously. Alternatively, clinical observation, such as an increase in cardiac output or a decrease in cardiac rate or both, may lead to a determination of increased efficiency. Cardiac efficiency can also be monitored by measurement of the amount of oxygen consumed per unit of exercise performed. In another example, improved efficiency of cardiac myocytes may be assessed by measurement of either or both the substrate consumed and the lactate produced per unit of exercise performed.

In a further aspect of the present invention, prophylactic and therapeutic methods are provided. Treatment on an acute or chronic basis is contemplated. In addition, treatment on an acute basis may be extended to chronic treatment, if so indicated. In one aspect, the present invention includes a method for the treatment or prevention of a condition associated with cardiac myocyte apoptosis in a subject in need thereof. The method generally comprises administering to the subject an amount of a GLP-1 molecule or agonist thereof effective to prevent or ameliorate apoptosis of cardiac myocytes, wherein the condition associated with cardiac myocyte apoptosis is thereby improved. As described herein, administration of any GLP-1 molecule or agonist thereof may be done in any manner and by any known GLP-1 molecule or agonist thereof.

In yet another embodiment of the invention, the methods of the present invention further comprise the identification of a subject in need of treatment. Any effective criteria may be used to determine that a subject may benefit from administration of a GLP-1 molecule or agonist thereof. Methods for the diagnosis of heart disease and diabetes, for example, as well as procedures for the identification of individuals at risk for development of these conditions, are well known to those in the art. Such procedures may include clinical tests, physical examination, personal interviews and assessment of family history.

B. GLP-1 Molecules of the Invention

In the context of the present invention, a GLP-1 molecule or agonist thereof includes any molecule with GLP-1 activity. In one embodiment, GLP-1 activity may be related to binding or activation of a GLP-1 receptor (e.g., a GLP-1 receptor agonist). A GLP-1 receptor is a cell-surface protein found, for example, on a cardiac myocyte. In this regard, a GLP-1 molecule agonist includes any molecule that binds to or activates a GLP-1 receptor.

Generally, GLP-1 receptor agonists can include peptides and small molecules, as known in the art. Exemplary GLP-1 receptor agonists have been described, such as those in Drucker, Endocrinology 144(12):5145-5148 (2003); EP 0708179; Hjorth et al., J. Biol. Chem. 269(48): 30121-30124 (1994); Siegel et al., Amer. Diabetes Assoc. 57^{th} Scientific Sessions, Boston (1997); Hareter et al., Amer. Diabetes Assoc. 57^{th} Scientific Sessions, Boston (1997); Adelhorst et al., J. Biol. Chem. 269(9): 6275-6278 (1994); Deacon et al., 16^{th} International Diabetes Federation Congress Abstracts, Diabetologia Supplement (1997); Irwin et al., Proc. Natl. Acad. Sci. USA. 94: 7915-7920 (1997); Mosjov, Int. J Peptide Protein Res. 40: 333-343 (1992); Göke et al., Diabetic Medicine 13: 854-860 (1996). Publications also disclose Black Widow GLP-1 and Ser² GLP-1. See Holz et al., Comparative Biochemistry and Physiology, Part B 121: 177-184 (1998) and Ritzel et al., “A synthetic glucagon-like peptide-1 analog with improved plasma stability,” J. Endocrinol. 159(1): 93-102 (1998).

In order to determine the ability of a GLP-1 molecule or agonist thereof to bind or activate a GLP-1 receptor, any available means can be used. In one embodiment, GLP-1 receptor binding or activation can be determined in either an in vitro or an in vivo model. In one embodiment, receptor-binding activity screening procedures may be used, such as for example, providing any cells that express GLP-1 receptor on the surface and measuring specific binding using radioimmunoassay methods. The cells expressing GLP-1 receptor can be naturally occurring or genetically modified. The cells expressing GLP-1 receptor may be cardiac myocyte cells. In one aspect, GLP-1 receptor binding or activation can be determined with the aid of combinatorial chemistry libraries and high throughput screening techniques, as is known in the art.

In one embodiment, GLP-1 molecule agonists that bind to or activate a GLP-1 receptor include exendin molecules, including exendin-1, exendin-2, exendin-3, exendin-4, and analogs thereof. Preferred exendin molecules include exendin-4 and analogs thereof. Such exendin molecules are generally known in the art and available to the skilled artisan.

By way of background, exendins are peptides that are found in the saliva of the Gila-monster, a lizard endogenous to Arizona, and the Mexican Beaded Lizard. Exendin-3 is present in the saliva of Heloderma horridum, and exendin-4 is present in the saliva of Heloderma suspectum (Eng, J., et al., J. Biol. Chem., 265:20259-62 (1990); Eng., J., et al., J. Biol. Chem., 267:7402-05 (1992)). The exendins have some sequence similarity to several members of the glucagon-like peptide family, with the highest identity, 53%, being to GLP-1 (Goke, et al., J. Biol. Chem., 268:19650-55 (1993)).

Exendin-4 is a potent agonist at GLP-1 receptors on insulin-secreting TC1 cells, at dispersed acinar cells from guinea pig pancreas, and at parietal cells from stomach; the peptide also stimulates somatostatin release and inhibits gastrin release in isolated stomachs (Goke, et al., J. Biol. Chem., 268:19650-55 (1993); Schepp, et al., Eur. J. Pharmacol., 69:183-91 (1994); Eissele, et al., Life Sci., 55:629-34 (1994)). Exendin-3 and exendin-4 were found to be GLP-1 agonists in stimulating cAMP production in, and amylase release from, pancreatic acinar cells (Malhotra, R., et al., Relulatory Peptides, 41:149-56 (1992); Raufman, et al., J. Biol. Chem., 267:21432-37 (1992); Singh, et al., Regul. Pept., 53:47-59 (1994)). The use of the insulinotropic activities of exendin-3 and exendin-4 for the treatment of diabetes mellitus and the prevention of hyperglycemia have been proposed (Eng, U.S. Pat. No. 5,424,286).

Truncated exendin peptides such as exendin[9-39], a carboxyamidated molecule, and fragments 3-39 through 9-39 have been reported to be potent and selective antagonists of GLP-1 (Goke, et al., J. Biol. Chem., 268:19650-55 (1993); Raufman, J. P., et al., J. Biol. Chem., 266:2897-902 (1991); Schepp, W., et al., Eur. J. Pharm., 269:183-91 (1994); Montrose-Rafizadeh, et al., Diabetes, 45(Suppl. 2):152A (1996)). Exendin[9-39] blocks endogenous GLP-1 in vivo, resulting in reduced insulin secretion (Wang, et al., J. Clin. Invest., 95:417-21 (1995); D'Alessio, et al., J. Clin. Invest., 97:133-38 (1996)). The receptor apparently responsible for the insulinotropic effect of GLP-1 has been cloned from rat pancreatic islet cells (Thorens, B., Proc. Natl. Acad. Sci. USA 89:8641-8645 (1992)). Exendins and exendin[9-39] bind to the cloned GLP-1 receptor (rat pancreatic -cell GLP-1 receptor: Fehmann HC, et al., Peptides, 15 (3): 453-6 (1994); human GLP-1 receptor: Thorens B, et al., Diabetes, 42 (11): 1678-82 (1993)). In cells transfected with the cloned GLP-1 receptor, exendin-4 is an agonist, i.e., it increases cAMP, while exendin[9-39] is an antagonist, i.e., it blocks the stimulatory actions of exendin-4 and GLP-1. Id.

In one embodiment an exendin analog can have one or more amino acid substitutions, deletions, inversion, or additions compared to a native or naturally occurring exendin. Thus, exendins analogs can have an amino acid sequence that has one or more amino acid substitutions, additions or deletions as compared with a naturally occurring exendin, for example, exendin-4. In one embodiment, an exendin analog has an amino acid sequence that has about 30 or less, 25 or less, 20 or less, 15 or less, 10 or less, 5 or less, 4 or less, 3 or less, 2 or less, or 1 or less substitutions, additions, or deletions as compared to a naturally occurring exendin, such as exendin-4.

Certain exendin compounds useful in the present invention include those disclosed in PCT/US98/16387, PCT/US98/24210, and PCT/US98/24273, and their corresponding U.S. applications Ser. Nos. 10/181,102, 09/554,533, and 09/554,531, respectively, all of which are herein incorporated by reference in their entireties. More particularly, exendin compounds include exendin peptide analogs in which one or more naturally occurring amino acids are eliminated or replaced with another amino acid(s). Particular exendin compounds are agonist analogs of exendin-4. In addition to exendin-3 [His Ser Asp Gly Thr Phe Thr Ser Asp Leu Ser Lys Gln Met Glu Glu Glu Ala Val Arg Leu Phe Ile Glu Trp Leu Lys Asn Gly Gly Pro Ser Ser Gly Ala Pro Pro Pro Ser], and exendin-4 [His Gly Glu Gly Thr Phe Thr Ser Asp Leu Ser Lys Gln Met Glu Glu Glu Ala Val Arg Leu Phe Ile Glu Trp Leu Lys Asn Gly Gly Pro Ser Ser Gly Ala Pro Pro Pro Ser], useful exendin compounds include exendin-4 (1-30) [His Gly Glu Gly Thr Phe Thr Ser Asp Leu Ser Lys Gln Met Glu Glu Glu Ala Val Arg Leu Phe Ile Glu Trp Leu Lys Asn Gly Gly], exendin-4 (1-30) amide [His Gly Glu Gly Thr Phe Thr Ser Asp Leu Ser Lys Gln Met Glu Glu Glu Ala Val Arg Leu Phe Ile Glu Trp Leu Lys Asn Gly Gly-NH₂], exendin-4 (1-28) amide [His Gly Glu Gly Thr Phe Thr Ser Asp Leu Ser Lys Gln Met Glu Glu Glu Ala Val Arg Leu Phe Ile Glu Trp Leu Lys Asn-NH₂], ¹⁴Leu,²⁵Phe exendin-4 [His Gly Glu Gly Thr Phe Thr Ser Asp Leu Ser Lys Gln Leu Glu Glu Glu Ala Val Arg Leu Phe Ile Glu Phe Leu Lys Asn Gly Gly Pro Ser Ser Gly Ala Pro Pro Pro Ser-NH₂], ¹⁴Leu,²⁵Phe exendin-4 (1-28) amide [His Gly Glu Gly Thr Phe Thr Ser Asp Leu Ser Lys Gln Leu Glu Glu Glu Ala Val Arg Leu Phe Ile Glu Phe Leu Lys Asn-NH₂], and ¹⁴Leu,²² Ala,²⁵Phe exendin-4 (1-28) amide [His Gly Glu Gly Thr Phe Thr Ser Asp Leu Ser Lys Gln Leu Glu Glu Glu Ala Val Arg Leu Ala Ile Glu Phe Leu Lys Asn-NH₂], and those described in International Application No. PCT/US98/16387, filed Aug. 6, 1998, entitled, “Novel Exendin Agonist Compounds,” and its corresponding U.S. application Ser. No. 10/181,102, including compounds of the formula (I):
Xaa₁ Xaa₂ Xaa₃ Gly Thr Xaa₄ Xaa₅ Xaa₆ Xaa₇ Xaa₈ Ser Lys Gln Xaag Glu Glu Glu Ala Val Arg Leu Xaa₁₀ Xaa₁₁ Xaa₁₂ Xaa₁₃ Leu Lys Asn Gly Gly Xaa₁₄ Ser Ser Gly Ala Xaa₁₅ Xaa₁₆ Xaa₁₇ Xaa₁₈-Z

wherein Xaa₁ is His, Arg or Tyr; Xaa₂ is Ser, Gly, Ala or Thr; Xaa₃ is Asp or Glu; Xaa₄ is Phe, Tyr or naphthylalanine; Xaa₅ is Thr or Ser; Xaa₆ is Ser or Thr; Xaa₇ is Asp or Glu; Xaa₈ is Leu, Ile, Val, pentylglycine or Met; Xaa₉ is Leu, Ile, pentylglycine, Val or Met; Xaa₁₀ is Phe, Tyr or naphthylalanine; Xaa₁₁ is Ile, Val, Leu, pentylglycine, tert-butylglycine or Met; Xaa₁₂ is Glu or Asp; Xaa₁₃ is Trp, Phe, Tyr, or naphthylalanine; Xaa₁₄, Xaa₁₅, Xaa₁₆ and Xaa₁₇ are independently Pro, homoproline, 3Hyp, 4Hyp, thioproline, N-alkylglycine, N-alkylpentylglycine or N-alkylalanine; Xaa₁₈ is Ser, Thr or Tyr; and Z is —OH or —NH₂; with the proviso that the compound is not exendin-3 or exendin-4.

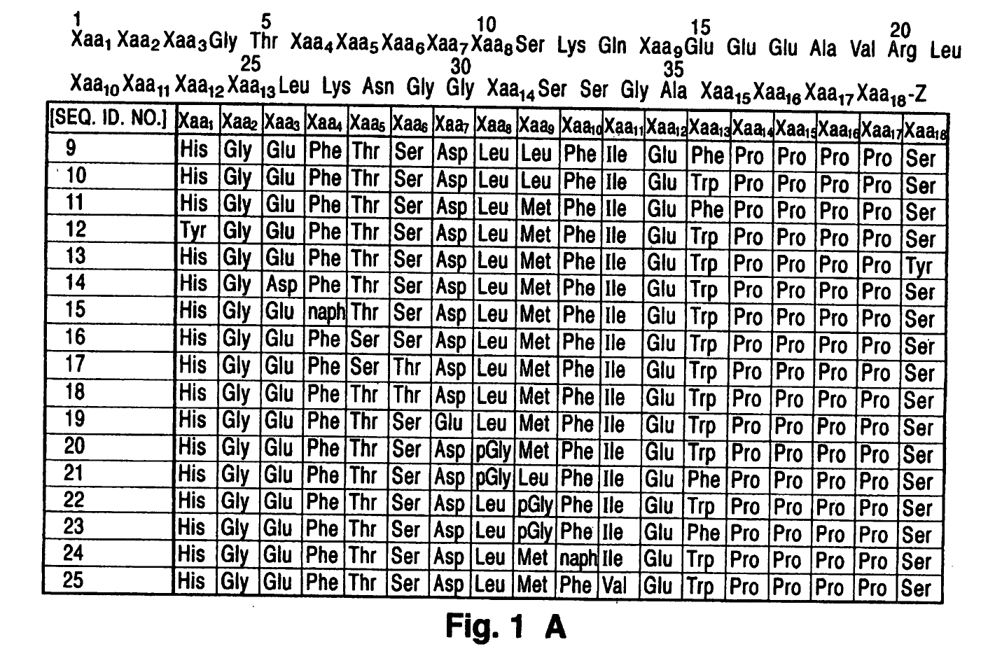

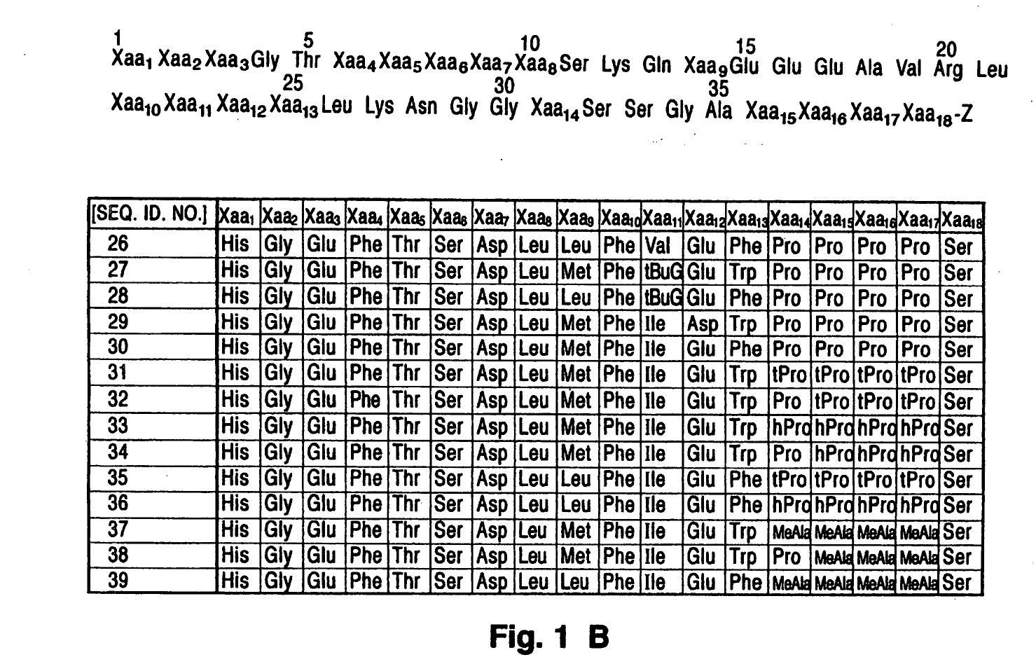

With reference to formula (I), preferred N-alkyl groups for N-alkylglycine, N-alkylpentylglycine and N-alkylalanine include lower alkyl groups of 1 to about 6 carbon atoms, or of 1 to 4 carbon atoms. Suitable compounds include those listed in FIGS. 1A and 1B.

Exemplary exendin compounds of formula (I) include those wherein Xaa₁ is His or Tyr, for example where Xaa₁ is His.

Included are those compounds of formula (I) wherein Xaa₂ is Gly.

Included are those compounds of formula (I) wherein Xaa₉ is Leu, pentylglycine, or Met.

Compounds of formula (I) include those wherein Xaa₁₃ is Trp or Phe.

Also included are compounds of formula (I) where Xaa₄ is Phe or naphthylalanine; Xaa₁₁ is Ile or Val and Xaa₁₄, Xaa₁₅, Xaa₁₆ and Xaa₁₇ are independently selected from Pro, homoproline, thioproline or N-alkylalanine. In one embodiment N-alkylalanine has a N-alkyl group of 1 to about 6 carbon atoms.

According to one aspect, compounds of formula (I) include those where Xaa₁₅, Xaa₁₆ and Xaa₁₇ are the same amino acid residue.

Included are compounds of formula (I) wherein Xaa₁₈ is Ser or Tyr, for example Ser.

With reference to formula (I), preferably Z is —NH2.

According to one aspect, included are compounds of formula (I) wherein Xaa₁ is His or Tyr, more preferably His; Xaa₂ is Gly; Xaa₄ is Phe or naphthylalanine; Xaa₉ is Leu, pentylglycine or Met; Xaa₁₀ is Phe or naphthylalanine; Xaa₁₁ is Ile or Val; Xaa₁₄, Xaa₁₅, Xaa16 and Xaa₁₇ are independently selected from Pro, homoproline, thioproline or N-alkylalanine; and Xaa₁₈ is Ser or Tyr, more preferably Ser. More preferably Z is —NH₂.

According to another aspect, compounds include those of formula (I) wherein: Xaa₁ is His or Arg; Xaa₂ is Gly; Xaa₃ is Asp or Glu; Xaa₄ is Phe or napthylalanine; Xaa₅ is Thr or Ser; Xaa6 is Ser or Thr; Xaa₇ is Asp or Glu; Xaa₈ is Leu or pentylglycine; Xaa₉ is Leu or pentylglycine; Xaa₁₀ is Phe or naphthylalanine; Xaa₁₁ is Ile, Val or t-butyltylglycine; Xaa₁₂ is Glu or Asp; Xaa₁₃ is Trp or Phe; Xaa₁₄, Xaa₁₅, Xaa₁₆, and Xaa₁₇ are independently Pro, homoproline, thioproline, or N-methylalanine; Xaa₁₈ is Ser or Tyr: and Z is —OH or —NH₂; with the proviso that the compound does not have the formula of either SEQ. ID. NOS. 1 or 2. More preferably, Z is —NH₂. Particular compounds include those having the amino acid sequence of SEQ. ID. NOS. 9, 10, 21, 22, 23, 26, 28, 34, 35 and 39.

According to one aspect, provided are compounds of formula (I) where Xaa₉ is Leu, Ile, Val or pentylglycine, more preferably Leu or pentylglycine, and Xaa₁₃ is Phe, Tyr or naphthylalanine, more preferably Phe or naphthylalanine. These compounds will exhibit advantageous duration of action and be less subject to oxidative degradation, both in vitro and in vivo, as well as during synthesis of the compound.

Exendin compounds also include compounds of the formula (II):
Xaahd 1 Xaa₂ Xaa₃ Gly Xaa₅ Xaa6 Xaa₇ Xaa₈ Xaa₉ Xaa₁₀ Xaa₁₁ Xaa₁₂ Xaa₁₃ Xaa₁₄ Xaa₁₅ Xaa₁₆ Xaa₁₇ Ala Xaa₁₉ Xaa₂₀ Xaa₂₁ Xaa₂₂ Xaa₂₃ Xaa₂₄ Xaa₂₅ Xaa₂₆ Xaa₂₇ Xaa₂₈-Z₁; wherein

wherein: Xaa₁ is His, Arg or Tyr; Xaa₂ is Ser, Gly, Ala or Thr; Xaa₃ is Ala Asp or Glu; Xaa₅ is Ala or Thr; Xaa6 is Ala, Phe, Tyr or naphthylalanine; Xaa₇ is Thr or Ser; Xaa₈ is Ala, Ser or Thr; Xaa₉ is Asp or Glu; Xaa₁₀ is Ala, Leu, Ile, Val, pentylglycine or Met; Xaa₁₁ is Ala or Ser; Xaa₁₂ is Ala or Lys; Xaa₁₃ is Ala or Gln; Xaa₁₄ is Ala, Leu, Ile, pentylglycine, Val or Met; Xaa₁₅ is Ala or Glu; Xaa₁₆ is Ala or Glu; Xaa₁₇ is Ala or Glu; Xaa₁₉ is Ala or Val; Xaa₂₀ is Ala or Arg; Xaa₂₁ is Ala or Leu; Xaa₂₂ is Ala, Phe, Tyr or naphthylalanine; Xaa₂₃ is Ile, Val, Leu, pentylglycine, tert-butylglycine or Met; Xaa₂₄ is Ala, Glu or Asp; Xaa₂₅ is Ala, Trp, Phe, Tyr or naphthylalanine; Xaa₂₆ is Ala or Leu; Xaa₂₇ is Ala or Lys; Xaa₂₈ is Ala or Asn; Z₁, is —OH, —NH₂, Gly-Z₂, Gly Gly-Z₂, Gly Gly Xaa₃₁-Z₂, Gly Gly Xaa₃₁ Ser-Z₂, Gly Gly Xaa₃₁ Ser Ser-Z₂, Gly Gly Xaa₃₁ Ser Ser Gly-Z₂, Gly Gly Xaa₃₁ Ser Ser Gly Ala-Z₂, Gly Gly Xaa₃₁ Ser Ser Gly Ala Xaa₃₆-Z₂, Gly Gly Xaa₃₁ Ser Ser Gly Ala Xaa₃₆ Xaa₃₇-Z₂ or Gly Gly Xaa₃₁ Ser Ser Gly Ala Xaa₃₆ Xaa₃₇ Xaa₃₈-Z₂; Xaa₃₁, Xaa₃₆, Xaa₃₇ and Xaa₃₈ are independently Pro, homoproline, 3Hyp, 4Hyp, thioproline, N-alkylglycine, N-alkylpentylglycine or N-alkylalanine; and Z₂ is —OH or —NH₂; provided that no more than three of Xaa₃, Xaa₅, Xaa₆, Xaa₈, Xaa₁₀, Xaa₁₁, Xaa₁₂, Xaa₁₃, Xaa₁₄, Xaa₁₅, Xaa₁₆, Xaa₁₇, Xaa₁₉, Xaa₂₀, Xaa₂₁, Xaa₂₄, Xaa₂₅, Xaa₂₆, Xaa₂₇ and Xaa₂₈ are Ala.

With reference to formula (II), N-alkyl groups for N-alkylglycine, N-alkylpentylglycine and N-alkylalanine include lower alkyl groups preferably of 1 to about 6 carbon atoms, more preferably of 1 to 4 carbon atoms.

Exendin compounds of formula (II) include those wherein Xaa₁ is His or Tyr. More preferably Xaa₁ is His.

Provided are those compounds of formula (II) wherein Xaa₂ is Gly.

Also provided are those compounds of formula (II) wherein Xaa₄ is Leu, pentylglycine or Met.

Exemplary compounds of formula (II) are those wherein Xaa₂₅ is Trp or Phe.

Exemplary compounds of formula (II) are those where Xaa6 is Phe or naphthylalanine; Xaa₂₂ is Phe or naphthylalanine and Xaa₂₃ is Ile or Val.

Provided are compounds of formula (II) wherein Xaa₃₁, Xaa₃₆, Xaa₃₇ and Xaa₃₈ are independently selected from Pro, homoproline, thioproline and N-alkylalanine.

With reference to formula (II), in one embodiment Z₁ is —NH₂.

With reference to formula (II), in one embodiment Z₂ is —NH₂.

According to one aspect, provided are compounds of formula (II) wherein Xaa₁ is His or Tyr, more preferably His; Xaa₂ is Gly; Xaa6 is Phe or naphthylalanine; Xaa₁₄ is Leu, pentylglycine or Met; Xaa₂₂ is Phe or naphthylalanine; Xaa₂₃ is Ile or Val; Xaa₃₁, Xaa₃₆, Xaa₃₇ and Xaa₃₈ are independently selected from Pro, homoproline, thioproline or N-alkylalanine. More preferably Z₁ is —NH₂.

According to a particular aspect, compounds include those of formula (II) wherein: Xaa₁ is His or Arg; Xaa₂ is Gly or Ala; Xaa₃ is Asp or Glu; Xaa₅ is Ala or Thr; Xaa6 is Ala, Phe or naphthylalaine; Xaa₇ is Thr or Ser; Xaa₈ is Ala, Ser or Thr; Xaag is Asp or Glu; Xaa₁₀ is Ala, Leu or pentylglycine; Xaa₁₁ is Ala or Ser; Xaa₁₂ is Ala or Lys; Xaa₁₃ is Ala or Gln; Xaa₁₄ is Ala, Leu or pentylglycine; Xaa₁₅ is Ala or Glu; Xaa₁₆ is Ala or Glu; Xaa₁₇ is Ala or Glu; Xaa₁₉ is Ala or Val; Xaa₂₀ is Ala or Arg; Xaa₂₁ is Ala or Leu; Xaa₂₂ is Phe or naphthylalanine; Xaa₂₃ is Ile, Val or tert-butylglycine; Xaa₂₄ is Ala, Glu or Asp; Xaa₂₅ is Ala, Trp or Phe; Xaa₂₆ is Ala or Leu; Xaa₂₇ is Ala or Lys; Xaa₂₈ is Ala or Asn; Z₁ is —OH, —NH₂, Gly-Z₂, Gly Gly-Z₂, Gly Gly Xaa₃₁-Z₂, Gly Gly Xaa₃₁ Ser-Z₂, Gly Gly Xaa₃₁ Ser Ser-Z₂, Gly Gly Xaa₃₁ Ser Ser Gly-Z₂, Gly Gly Xaa₃₁ Ser Ser Gly Ala-Z₂, Gly Gly Xaa₃₁ Ser Ser Gly Ala Xaa₃₆-Z₂, Gly Gly Xaa₃₁ Ser Ser Gly Ala Xaa₃₆ Xaa₃₇-Z₂, Gly Gly Xaa₃₁ Ser Ser Gly Ala Xaa₃₆ Xaa₃₇ Xaa₃₈-Z₂; Xaa₃₁, Xaa₃₆, Xaa₃₇ and Xaa₃₈ being independently Pro homoproline, thioproline or N-methylalanine; and Z₂ being —OH or —NH₂; provided that no more than three of Xaa₃, Xaa₅, Xaa6, Xaa₈, Xaa₁₀, Xaa₁₁, Xaa₁₂, Xaa₁₃, Xaa₁₄, Xaa₁₅, Xaa₁₆, Xaa₁₇, Xaa₁₉, Xaa₂₀, Xaa₂₁, Xaa₂₄, Xaa₂₅, Xaa₂₆, Xaa₂₇ and Xaa₂₈ are Ala. Exemplary compounds include those having the amino acid sequence of SEQ. ID. NOS. 40-61.

According to one aspect, provided are compounds of formula (II) where Xaa₁₄ is Leu, Ile, Val or pentylglycine, more preferably Leu or pentylglycine, and Xaa₂₅ is Phe, Tyr or naphthylalanine, more preferably Phe or naphthylalanine. These compounds will be less susceptive to oxidative degradation, both in vitro and in vivo, as well as during synthesis of the compound.

Exendin compounds also include compounds of the formula (III):
Xaa₁ Xaa₂ Xaa₃ Xaa₄ Xaa₅ Xaa6 Xaa₇ Xaa₈ Xaa₉ Xaa₁₀ Xaa₁₁ Xaa₁₂ Xaa₁₃ Xaa₁₄ Xaa₁₅ Xaa₁₆ Xaa₁₇ Ala Xaa₁₉ Xaa₂₀ Xaa₂₁ Xaa₂₂ Xaa₂₃ Xaa₂₄ Xaa₂₅ Xaa₂₆ Xaa₂₇ Xaa₂₈-Z₁; wherein

wherein: Xaa₁ is His, Arg, Tyr, Ala, Norval, Val, or Norleu; Xaa₂ is Ser, Gly, Ala or Thr; Xaa₃ is Ala, Asp or Glu; Xaa₄ is Ala, Norval, Val, Norleu or Gly; Xaa₅ is Ala or Thr; Xaa₆ is Ala, Phe, Tyr or naphthylalanine; Xaa₇ is Thr or Ser; Xaa₈ is Ala, Ser or Thr; Xaa₉ is Ala, Norval, Val, Norleu, Asp or Glu; Xaa₁₀ is Ala, Leu, Ile, Val, pentylglycine or Met; Xaa₁₁ is Ala or Ser; Xaa₁₂ is Ala or Lys; Xaa₁₃ is Ala or Gln; Xaa₁₄ is Ala, Leu, Ile, pentylglycine, Val or Met; Xaa₁₅ is Ala or Glu; Xaa₁₆ is Ala or Glu; Xaa₁₇ is Ala or Glu; Xaa₁₉ is Ala or Val; Xaa₂₀ is Ala or Arg; Xaa₂₁ is Ala or Leu; Xaa₂₂ is Phe, Tyr or naphthylalanine; Xaa₂₃ is Ile, Val, Leu, pentylglycine, tert-butylglycine or Met; Xaa₂₄ is Ala, Glu or Asp; Xaa₂₅ is Ala, Trp, Phe, Tyr or naphthylalanine; Xaa₂₆ is Ala or Leu; Xaa₂₇ is Ala or Lys; Xaa₂₈ is Ala or Asn; Z₁ is —OH, NH₂, Gly-Z₂, Gly Gly-Z₂, Gly Gly Xaa₃₁-Z₂, Gly Gly Xaa₃₁ Ser-Z₂, Gly Gly Xaa₃₁ Ser Ser-Z₂, Gly Gly Xaa₃₁ Ser Ser Gly-Z₂, Gly Gly Xaa₃₁ Ser Ser Gly Ala-Z₂, Gly Gly Xaa₃₁ Ser Ser Gly Ala Xaa₃₆-Z₂, Gly Gly Xaa₃₁ Ser Ser Gly Ala Xaa₃₆ Xaa₃₇-Z₂, Gly Gly Xaa₃₁ Ser Ser Gly Ala Xaa₃₆ Xaa₃₇ Xaa₃₈-Z₂ or Gly Gly Xaa₃₁ Ser Ser Gly Ala Xaa₃₆ Xaa₃₇ Xaa₃₈ Xaa₃₉-Z₂; wherein Xaa₃₁, Xaa₃₆, Xaa₃₇ and Xaa₃₈ are independently Pro, homoproline, 3Hyp, 4Hyp, thioproline, N-alkylglycine, N-alkylpentylglycine or N-alkylalanine; Xaa₃₉ is Ser, Thr, Lys or Ala; and Z₂ is —OH or —NH₂; provided that no more than three of Xaa₃, Xaa₄, Xaa₅, Xaa₆, Xaa₈, Xaa₉, Xaa₁₀, Xaa₁₁, Xaa₁₂, Xaa₁₃, Xaa₁₄, Xaa₁₅, Xaa₁₆, Xaa₁₇, Xaa₁₉, Xaa₂₀, Xaa₂₁, Xaa₂₄, Xaa₂₅, Xaa₂₆, Xaa₂₇ and Xaa₂₈ are Ala; and provided also that, if Xaa₁ is His, Arg or Tyr, then at least one of Xaa₃, Xaa₄ and Xaa₉ is Ala.

In another embodiment, GLP-1 molecules include GLP-1 peptides. By way of non-limiting example, a GLP-1 peptide includes GLP-1 (1-37), GLP-1 (1-36) amide, GLP-1 (7-37), and GLP-1 (7-36) amide (known in the art as “GLP-1”). In one embodiment, a GLP-1 peptide used in the methods of the present invention is a long-acting GLP-1 analog. A long acting analog refers to any GLP-1 molecule that has a longer in vivo half-life than GLP-1. Such long-acting GLP-1 analogs are known in the art and described herein.

A GLP-1 molecule also includes any biologically active analogs, including variants and derivatives, of GLP-1 peptides. A biologically active GLP-1 analog, including a variant or derivative thereof, can possess GLP-1 biological activity that is more potent, less potent or equally potent as compared to the biological activity of a native GLP-1. A biologically active GLP-1 analog also includes those molecules that can exhibit GLP-1 activity upon cleavage, translation, or any other processing that occurs upon administration of the GLP-1 molecule.

In an embodiment, a GLP-1 analog includes any peptides that are formed by conservative amino acid substitution of a GLP-1 peptide. For example, it is well known in the art that one or more amino acids in a sequence, such as an amino acid sequence for GLP-1, can be substituted with other amino acid(s), the charge and polarity of which are similar to that of the native amino acid. Hydropathic index of amino acids can be considered when making amino acid changes. The importance of the hydropathic amino acid index in conferring interactive biological function on a protein is generally understood in the art (Kyte and Doolittle, J. Mol. Biol. 157:105-132 (1982)). It is also understood in the art that the conservative substitution of amino acids can be made effectively on the basis of hydrophilicity. U.S. Pat. No. 4,554,101 states that the greatest local average hydrophilicity of a protein, as governed by the hydrophilicity of its adjacent amino acids, correlates with a biological property of the protein. In making such changes, the substitution of amino acids whose hydrophilicity values are within ±2 is preferred, those that are within ±1 are particularly preferred, and those within ±0.5 are even more particularly preferred.

Due to the degeneracy of the genetic code, different nucleotide codons can encode a particular amino acid. Accordingly, the present invention contemplates that a nucleic acid molecule encoding a GLP-1 molecule can have any codon usage that encodes a GLP-1 molecule. A host cell often exhibits a preferred pattern of codon usage. In a preferred embodiment, the codon usage of a nucleotide sequence encoding a GLP-1 reflects a preferred codon usage for a host in which the GLP-1 molecule will be used.

In another embodiment, a GLP-1 analog has an amino acid sequence that has one or more amino acid substitutions, additions or deletions as compared with a GLP-1 peptide, for example GLP-1. In one embodiment, a GLP-1 analog has an amino acid sequence that has about 30 or less, 25 or less, 20 or less, 15 or less, 10 or less, 5 or less, 4 or less, 3 or less, 2 or less, or 1 or less substitutions, additions, or deletions as compared to a GLP-1 peptide. Various GLP-1 analogs are generally known in the art and are available to the skilled artisan.

In another embodiment, a GLP-1 analog has at least 60%, at least 70%, at least 80%, at least 90% or at least 95% sequence identity with a naturally occurring GLP-1. Identity, as is well understood in the art, is a relationship between two or more polypeptide sequences or two or more polynucleotide sequences, as determined by comparing the sequences. In the art, identity also means the degree of sequence relatedness between polypeptide or polynucleotide sequences, as determined by the match between strings of such sequences. Identity can be readily calculated by known methods including, but not limited to, those described in Computational Molecular Biology, Lesk, A. M., ed., Oxford University Press, New York (1988); Biocomputing: Informatics and Genome Projects, Smith, D. W., ed., Academic Press, New York, 1993; Computer Analysis of Sequence Data, Part I, Griffin, A. M. and Griffin, H. G., eds., Humana Press, New Jersey (1994); Sequence Analysis in Molecular Biology, von Heinje, G., Academic Press (1987); Sequence Analysis Primer, Gribskov, M. and Devereux, J., eds., Stockton Press, New York (1991); and Carillo, H., and Lipman, D., SIAM J Applied Math, 48:1073 (1988). Methods to determine identity are designed to give the largest match between the sequences tested. Moreover, methods to determine identity are codified in publicly available programs. Computer programs which can be used to determine identity between two sequences include, but are not limited to, GCG (Devereux, J., et al., Nucleic Acids Research 12(1):387 (1984); suite of five BLAST programs, three designed for nucleotide sequences queries (BLASTN, BLASTX, and TBLASTX) and two designed for protein sequence queries (BLASTP and TBLASTN) (Coulson, Trends in Biotechnology, 12: 76-80 (1994); Birren, et al., Genome Analysis, 1: 543-559 (1997)). The BLAST X program is publicly available from NCBI and other sources (BLAST Manual, Altschul, S., et al., NCBI NLM NIH, Bethesda, Md. 20894; Altschul, S., et al., J. Mol. Biol., 215:403-410 (1990)). The well known Smith Waterman algorithm can also be used to determine identity.

More particularly, as used herein, a “GLP-1 analog” is defined as a molecule having one or more amino acid substitutions, deletions, inversions, or additions compared with a native GLP-1 peptide. A “GLP-1 derivative” is defined as a molecule having the amino acid sequence of a native GLP-1 peptide or of a GLP-1 analog, but additionally having chemical modification of one or more of its amino acid side groups, alpha.-carbon atoms, terminal amino group, or terminal carboxylic acid group. A chemical modification includes, but is not limited to, adding chemical moieties, creating new bonds, and removing chemical moieties. Modifications at amino acid side groups include, without limitation, acylation of lysine .epsilon.-amino groups, N-alkylation of arginine, histidine, or lysine, alkylation of glutamic or aspartic carboxylic acid groups, and deamidation of glutamine or asparagine. Modifications of the terminal amino include, without limitation, the desamino, N-lower alkyl, N-di-lower alkyl, and N-acyl modifications. Modifications of the terminal carboxy group include, without limitation, the amide, lower alkyl amide, dialkyl amide, and lower alkyl ester modifications. Lower alkyl is C1-C4 alkyl. Furthermore, one or more side groups, or terminal groups, may be protected by protective groups known to the ordinarily-skilled protein chemist. The α-carbon of an amino acid may be mono- or dimethylated.

GLP-1 analogs known in the art include, for example, GLP-1(7-34) and GLP-1(7-35), Gln⁹-GLP-1(7-37), D-Gln⁹-GLP-1(7-37), Thr¹⁶-Lys¹⁸⁻GLP-1(7-37), and Lys¹⁸-GLP-1(7-37). Other preferred GLP-1 analogs include: Gly⁸-GLP-1 (7-36)NH₂, Gln⁹-GLP-1 (7-37), D-Gln⁹-GLP-1 (7-37), acetyl-Lys⁹-GLP-1(7-37), Thr⁹-GLP-1(7-37), D-Thr⁹-GLP-1 (7-37), Asn⁹-GLP-1 (7-37), D-Asn⁹-GLP-1 (7-37), Ser²²-Arg²³-Arg²⁴-Gln²⁶-GLP-1(7-37), Thr¹⁶-Lys¹⁸-GLP-1(7-37), Lys¹⁸-GLP-1(7-37), Arg²³-GLP-1(7-37), Arg²⁴-GLP-1(7-37), and the like (see, e.g., WO 91/11457).

Other GLP-1 analogs are disclosed in U.S. Pat. No. 5,545,618 which is incorporated herein by reference. A preferred group of GLP-1 analogs and derivatives include those disclosed in U.S. Pat. No. 6,747,006, which is herein incorporated by reference in its entirety. The use in the present invention of a molecule described in U.S. Pat. No. 5,188,666, which is expressly incorporated by reference, is also contemplated. Another group of molecules for use in the present invention includes compounds described in U.S. Pat. No. 5,512,549, which is expressly incorporated herein by reference.

Another group of active compounds for use in the present invention is disclosed in WO 91/11457, and consists essentially of GLP-1(7-34), GLP-1(7-35), GLP-1(7-36), or GLP-1(7-37), or the amide form thereof, and pharmaceutically-acceptable salts thereof, having at least one modification selected from the group consisting of:

(a) substitution of glycine, serine, cysteine, threonine, asparagine, glutamine, tyrosine, alanine, valine, isoleucine, leucine, methionine, phenylalanine, arginine, or D-lysine for lysine at position 26 and/or position 34; or substitution of glycine, serine, cysteine, threonine, asparagine, glutamine, tyrosine, alanine, valine, isoleucine, leucine, methionine, phenylalanine, lysine, or a D-arginine for arginine at position 36;

(b) substitution of an oxidation-resistant amino acid for tryptophan at position 31;

(c) substitution of at least one of: tyrosine for valine at position 16; lysine for serine at position 18; aspartic acid for glutamic acid at position 21; serine for glycine at position 22; arginine for glutamine at position 23; arginine for alanine at position 24; and glutamine for lysine at position 26; and

(d) substitution of at least one of: glycine, serine, or cysteine for alanine at position 8; aspartic acid, glycine, serine, cysteine, threonine, asparagine, glutamine, tyrosine, alanine, valine, isoleucine, leucine, methionine, or phenylalanine for glutamic acid at position 9; serine, cysteine, threonine, asparagine, glutamine, tyrosine, alanine, valine, isoleucine, leucine, methionine, or phenylalanine for glycine at position 10; and glutamic acid for aspartic acid at position 15; and

(e) substitution of glycine, serine, cysteine, threonine, asparagine, glutamine, tyrosine, alanine, valine, isoleucine, leucine, methionine, or phenylalanine, or the D- or N-acylated or alkylated form of histidine for histidine at position 7; wherein, in the substitutions is (a), (b), (d), and (e), the substituted amino acids can optionally be in the D-form and the amino acids substituted at position 7 can optionally be in the N-acylated or N-alkylated form.

Because the enzyme, dipeptidyl-peptidase IV (DPP IV), may be responsible for the observed rapid in vivo inactivation of administered GLP-1, (see, e.g., Mentlein, R., et al., Eur. J. Biochem., 214:829-835 (1993)), administration of GLP-1 analogs and derivatives that are protected from the activity of DPP IV is preferred, and the administration of Gly⁸-GLP-1(7-36)NH₂, Val⁸-GLP-1(7-37)OH, α-methyl-Ala⁸-GLP-1(7-36)NH₂, and Gly⁸-Gln²¹-GLP-1(7-37)OH, or pharmaceutically-acceptable salts thereof, is more preferred.

A GLP-1 molecule or agonist thereof can be obtained from any source. In one embodiment, a GLP-1 molecule or agonist thereof can be obtained from an organism, such as a mouse, a rat, a lizard, or a human. It is also contemplated herein that a GLP-1 molecule or agonist thereof can be obtained by any method or combination of methods known to the skilled artisan. In an illustrative embodiment, a GLP-1 molecule can be isolated by collection of a secretion, by extraction, by purification, or by any combination such of methods. In another embodiment, a GLP-1 molecule can be identified and purified by the use of monoclonal, polyclonal, or any combination of antibodies. Antibodies such as ABGA1178 detect intact, unspliced GLP-1 (1-37) or N-terminally truncated GLP-1 (7-37) or GLP-1. In addition, other antibodies detect at the very end of the C-terminus of the precursor molecule (See e.g., Osrkov et al., J. Clin. Invest. 87: 415-423 (1991)).

In another embodiment, GLP-1 or agonists thereof can be obtained by any recombinant means. A recombinant GLP-1 molecule or agonist thereof includes any molecule that is, or results, however indirectly, from human manipulation of a nucleic or amino acid molecule. In one embodiment, a recombinant molecule is a recombinant human peptide.

In yet another embodiment, a GLP-1 molecule agonist may be a small molecule which binds or activates a GLP-1 receptor, and may be synthesized in any manner known in the art.

In another embodiment, the use of DPP IV inhibitors to decrease or eliminate the inactivation of endogenous GLP-1 is also contemplated. DPP IV inhibitors can be administered alone or in combination with a GLP-1 molecule or agonist thereof. As such, it is contemplated that active GLP-1 molecules may be increased by the inhibition of DPP IV. Inhibitors of DPP IV are known to the skilled artisan and include, by way of non-limiting example, 2-cyanopyrrolidines. See e.g., Fukushima, H., et al., Bioorg. Med. Chem. Lett. 14(22): 6053-6061 (2004). Non-limiting exemplary DPP IV inhibitors include valine-pyrrolidide (Marguet, D., et al., Proc. Natl. Acad. Sci. USA 97(12): 6874-6879 (2000)), isoleucine thiazolidide (Pederson, R. A., et al., Diabetes 47: 1253-1258 (1998), and NVP-DPP728 (Balkan, B., et al., Diabetologia 42(11):1324-1331 (1999)). DPP IV inhibitors including ketopyrrolidines and ketoazetidines have been discussed in the literature (Ferraris, D., et al., Bioorg. Med. Chem. Lett. 14(22): 5579-5583 (2004)). Metformin and pioglitazone have been proposed to reduce DPP IV activity in vivo (Kenhard, J. M., et al., Biochem. Biophys. Res. Commun. 324(1):92-97 (2004). Literature reports further describe optimization of a proline derived homophenylalanine 3 to produce a potent DPP IV inhibitor. See Edmondson, S. D., et al., Bioorg. Med. Chem. Lett. 14(20): 5151-5155 (2004).

C. Pharmaceutical Compositions of the Invention

The GLP-1 molecules or agonists thereof may be formulated as pharmaceutical compositions for use in conjunction with the methods of the present invention. The pharmaceutical compositions may be formulated with pharmaceutically acceptable excipients such as carriers, solvents, stabilizers, adjuvants, diluents, etc., depending upon the particular mode of administration and dosage form. The pharmaceutical compositions should generally be formulated to achieve a physiologically compatible pH, and may range from a pH of about 3 to a pH of about 11, or from about pH 3 to about pH 7, depending on the formulation and route of administration. In alternative embodiments, the pH may be adjusted to a range from about pH 5.0 to about pH 8.0 or from about pH 4.0 to about pH 5.0.

In an embodiment, a pharmaceutical composition of the invention comprises an effective amount of at least one GLP-1 molecule or agonist thereof, together with one or more pharmaceutically acceptable excipients. Optionally, a pharmaceutical composition may include a second active ingredient useful in the prevention of cardiac myocyte apoptosis.

The pharmaceutical compositions may be formulated for administration in any manner known in the art. By way of example, when formulated for oral administration or parenteral administration, the pharmaceutical composition is most typically a solid, liquid solution, emulsion or suspension, while inhaleable formulations for pulmonary or nasal administration are generally liquids or powders. A pharmaceutical composition may also be formulated as a lyophilized solid that is reconstituted with a physiologically compatible solvent prior to administration. Alternative pharmaceutical compositions of the invention may be formulated as syrups, creams, ointments, tablets, and the like.

The term “pharmaceutically acceptable excipient” refers to an excipient for administration of a pharmaceutical agent, such as a GLP-1 molecule or agonist thereof. The term refers to any pharmaceutical excipient that may be administered without undue toxicity. Pharmaceutically acceptable excipients are determined in part by the particular composition being administered, as well as by the particular method used to administer the composition. Accordingly, there exists a wide variety of suitable formulations of pharmaceutical compositions for use in the methods of the present invention (see, e.g., Remington's Pharmaceutical Sciences).

Suitable excipients may be carrier molecules that include large, slowly metabolized macromolecules such as proteins, polysaccharides, polylactic acids, polyglycolic acids, polymeric amino acids, amino acid copolymers, and inactive virus particles. Other exemplary excipients include antioxidants such as ascorbic acid; chelating agents such as EDTA; carbohydrates such as dextrin, hydroxyalkylcellulose, hydroxyalkylmethylcellulose, stearic acid; liquids such as oils, water, saline, glycerol and ethanol; wetting or emulsifying agents; pH buffering substances; and the like. Liposomes are also included within the definition of pharmaceutically acceptable excipients.

More particularly, when intended for oral use, e.g., tablets, troches, lozenges, aqueous or oil suspensions, non-aqueous solutions, dispersible powders or granules (including micronized particles or nanoparticles), emulsions, hard or soft capsules, syrups or elixirs may be prepared. Compositions intended for oral use may be prepared according to any method known to the art for the manufacture of pharmaceutical compositions, and such compositions may contain one or more agents including sweetening agents, flavoring agents, coloring agents and preserving agents, in order to provide a palatable preparation.

Pharmaceutically acceptable excipients particularly suitable for use in conjunction with tablets include, for example, inert diluents, such as celluloses, calcium or sodium carbonate, lactose, calcium or sodium phosphate; disintegrating agents, such as croscarmellose sodium, cross-linked povidone, maize starch, or alginic acid; binding agents, such as povidone, starch, gelatin or acacia; and lubricating agents, such as magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

Formulations for oral use may be also presented as hard gelatin capsules where the active ingredient is mixed with an inert solid diluent, for example celluloses, lactose, calcium phosphate or kaolin, or as soft gelatin capsules wherein the active ingredient is mixed with non-aqueous or oil medium, such as glycerin, propylene glycol, polyethylene glycol, peanut oil, liquid paraffin or olive oil.

In another embodiment, the pharmaceutical composition of the invention may be formulated as a suspension comprising a GLP-1 molecule or agonist thereof in admixture with at least one pharmaceutically acceptable excipient suitable for the manufacture of a suspension. In yet another embodiment, a GLP-1 molecule or agonist thereof may be formulated as dispersible powder and granules suitable for preparation of a suspension by the addition of suitable excipients.

Excipients suitable for use in connection with suspensions include suspending agents, such as sodium carboxymethylcellulose, methylcellulose, hydroxypropyl methylcelluose, sodium alginate, polyvinylpyrrolidone, gum tragacanth, gum acacia, dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycethanol), a condensation product of ethylene oxide with a partial ester derived from a fatty acid and a hexitol anhydride (e.g., polyoxyethylene sorbitan monooleate); and thickening agents, such as carbomer, beeswax, hard paraffin or cetyl alcohol. The suspensions may also contain one or more preservatives such as acetic acid, methyl and/or n-propyl p-hydroxy-benzoate; one or more coloring agents; one or more flavoring agents; and one or more sweetening agents such as sucrose or saccharin.

The pharmaceutical composition of the present invention may also be in the form of an oil-in-water emulsion. The oily phase may be a vegetable oil, such as olive oil or arachis oil, a mineral oil, such as liquid paraffin, or a mixture of these. Suitable emulsifying agents include naturally-occurring gums, such as gum acacia and gum tragacanth; naturally occurring phosphatides, such as soybean lecithin, esters or partial esters derived from fatty acids; hexitol anhydrides, such as sorbitan monooleate; and condensation products of these partial esters with ethylene oxide, such as polyoxyethylene sorbitan monooleate. The emulsion may also contain sweetening and flavoring agents. Syrups and elixirs may be formulated with sweetening agents, such as glycerol, sorbitol or sucrose. Such formulations may also contain a demulcent, a preservative, a flavoring or a coloring agent.

In another embodiment, the pharmaceutical composition of the invention may be formulated as a sterile injectable preparation, such as a sterile injectable aqueous emulsion or oleaginous suspension. This emulsion or suspension may be formulated according to the known art using those suitable dispersing or wetting agents and suspending agents such as those that have been mentioned above. In another preferred embodiment, the sterile injectable preparation may also be a sterile injectable solution or suspension in a non-toxic parenterally acceptable diluent or solvent, such as a solution in 1,2-propane-diol. The sterile injectable preparation may also be prepared as a lyophilized powder. Among the acceptable vehicles and solvents that may be employed are water, Ringer's solution, and isotonic sodium chloride solution. In addition, sterile fixed oils may be employed as a solvent or suspending medium. For this purpose any bland fixed oil may be employed including synthetic mono- or diglycerides. In addition, fatty acids such as oleic acid may likewise be used in the preparation of injectables.

Certain GLP-1 molecules or agonists thereof may be substantially insoluble in water and sparingly soluble in most pharmaceutically acceptable protic solvents and in vegetable oils. However, the compounds may be soluble in medium chain fatty acids (e.g., caprylic and capric acids) or triglycerides and have high solubility in propylene glycol esters of medium chain fatty acids. Also contemplated for use in the methods of the invention are compositions, which have been modified by substitutions or additions of chemical or biochemical moieties which make them more suitable for delivery (e.g., increase solubility, bioactivity, palatability, decrease adverse reactions, etc.), for example by esterification, glycation, PEGylation, etc.

A GLP-1 molecule or agonist thereof may also be formulated for oral administration in a self-emulsifying drug delivery system (SEDDS). Lipid-based formulations such as SEDDS are particularly suitable for low solubility compounds, and can generally enhance the oral bioavailability of such compounds.

In an alternative embodiment, cyclodextrins may be added as aqueous solubility enhancers. Cyclodextrins include hydroxypropyl, hydroxyethyl, glucosyl, maltosyl and maltotriosyl derivatives of α-, β-, and γ-cyclodextrin. An exemplary cyclodextrin solubility enhancer is hydroxypropyl-β-cyclodextrin (HPBC), which may be added to any of the above-described compositions to further improve the aqueous solubility characteristics of a GLP-1 molecule or agonist thereof. In one embodiment, the composition comprises 0.1% to 20% hydroxypropyl-β-cyclodextrin, in another embodiment 1% to 15% hydroxypropyl-β-cyclodextrin, and in still another embodiment from 2.5% to 10% hydroxypropyl-β-cyclodextrin. The amount of solubility enhancer employed will depend on the amount of GLP-1 molecule or agonist thereof in the composition.

Dosage and administration are adjusted to provide sufficient levels of the active agent(s) in a pharmaceutical composition or to maintain the desired effect. Factors that may be taken into account include the severity of the disease state, general health of the subject, age, weight, and gender of the subject, diet, time and frequency of administration, drug combination(s), reaction sensitivities, and tolerance/response to therapy. Whether an administration is acute or chronic may also be considered in determining dosage. Long-acting pharmaceutical compositions may be administered every 3 to 4 days, every week, or once every two weeks depending on half-life and clearance rate of the particular formulation. In one embodiment, GLP-1 molecules or agonists thereof used in the methods of the present invention are administered continuously.

D. Combination Therapy

In another aspect of the invention, it is also possible to combine a GLP-1 molecule or agonist thereof useful in the methods of the present invention, with one or more other active ingredients useful in the prevention of cardiac myocyte apoptosis. For example, a GLP-1 molecule or agonist thereof may be combined with one or more other compounds, in a unitary dosage form, or in separate dosage forms intended for simultaneous or sequential administration to a patient in need of treatment. When administered sequentially, the combination may be administered in two or more administrations. In an alternative embodiment, it is possible to administer one or more GLP-1 molecules or agonists thereof and one or more additional active ingredients by different routes. The skilled artisan will also recognize that a variety of active ingredients may be administered in combination with GLP-1 molecules or agonists thereof that may act to augment or synergistically enhance the prevention of cardiac myocyte apoptosis.

According to the methods of the invention, a GLP-1 molecule or agonist thereof may be: (1) co-formulated and administered or delivered simultaneously in a combined formulation; (2) delivered by alternation or in parallel as separate formulations; or (3) by any other combination therapy regimen known in the art. When delivered in alternation therapy, the methods of the invention may comprise administering or delivering the active ingredients sequentially, e.g., in separate solution, emulsion, suspension, tablets, pills or capsules, or by different injections in separate syringes. In general, during alternation therapy, an effective dosage of each active ingredient is administered sequentially, i.e., serially, whereas in simultaneous therapy, effective dosages of two or more active ingredients are administered together. Various sequences of intermittent combination therapy may also be used.

### EXAMPLES

### Example 1

Cardiac Myocyte Isolation and Culture

For use in conjunction with the present invention, cardiac myocytes may be isolated as follows. Calcium-tolerant adult rat ventricular myocytes (ARVMs) are obtained from hearts of male Sprague-Dawley rats. Animals are euthanized with sodium pentobarbital (50 mg/kg IP) and heparinized (1000 USP/kg IV), and their hearts are aseptically removed into an ice-cold modified cardioplegic solution (KB solution, in mmol/L: KOH 85, KCl 30, KH₂PO₄ 30, MgSO₄ 3, EGTA 0.5, HEPES 10, L-glutamic acid 50, and taurine 20, at pH 7.4). The hearts are retrograde-perfused on a Langendorff apparatus with Tyrode's solution (in mmol/L: NaCl 137, KCl 5.4, CaCl₂ 1.2, MgCl₂ 0.5, HEPES 10, and glucose 10, at pH 7.4) for 5 minutes at 37° C. The perfusion solution is switched to a nominally Ca²⁺-free Tyrode's solution for 6 minutes and then to a nominally Ca²⁺-free Tyrode's solution containing 0.02% protease (Sigma) and 0.06% collagenase A (Boehringer Manheim). After 10 to 15 minutes, the enzymatic solution is washed out for an additional 5 minutes. After perfusion, cells from the left ventricle are released by shaking the tissue. The cells are filtered through a 15-nm mesh and allowed to settle (40 minutes) in KB solution. The cells are resuspended in DMEM (Gibco), layered over 60 μg/mL BSA (Sigma) to separate ventriclar myocytes from nonmyocytes as described in Ellington, and allowed to settle for 10 to 15 minutes (Ellington, Amer. J. Physiol. 265: H747-745 (1993)). Cells are resuspended in ACCT medium containing Dulbecco's Modified Eagle's Medium (DMEM) with 2 mg/mL BSA, 2 mmol/L L-carnitine, 5 mmol/L creatine, 5 mmol/L taurine, 100 IU/mL penicillin, and 100 μg/mL streptomycin. The ARVMs are plated in ACCT medium at a density of 100 to 150 cell/mm² on 100-mm or 35-mm plastic culture dishes (Fisher) or 40×22-mm glass coverslips (Fisher) precoated with laminin (1 mg/cm², Becton-Dickinson). After 1 hour, the dishes are washed with ACCT to remove cells that are not attached. The remaining cells are then be maintained in ACCT medium for approximately 16 plus hours before the addition of GLP-1 molecules and norepinephrine (to stimulate apoptosis).

### Example 2

GLP-1 Receptor Binding Assay

GLP-1 receptor binding activity and affinity may be measured using a binding displacement assay in which the receptor source is RINm5F cell membranes, and the ligand is [¹²⁵I]GLP-1. Homogenized RINm5F cell membranes are incubated in 20 mM HEPES buffer with 40,000 cpm [¹²⁵I]GLP-1 tracer, and varying concentrations of test compound for 2 hours at 23° C. with constant mixing. Reaction mixtures are filtered through glass filter pads presoaked with 0.3% PEI solution and rinsed with ice-cold phosphate buffered saline. Bound counts are determined using a scintillation counter. Binding affinities are calculated using GraphPad Prism software (GraphPad Software, Inc., San Diego, Calif.).

The following results are obtained:

| | | Standard |
|---|---|---|
| Name | IC₅₀ (nM) | Deviation |
| GLP-1 (9-36) | 65 | 0 |
| GLP-1 (7-36) | 0.152 | 0.033 |
| Exendin-4 | 0.53 | 0.122 |

### Example 3

Apoptosis Assays

A. Detection of DNA Fragmentation:

Internucleosomal cleavage of DNA may be analyzed by the presence of DNA laddering on agarose gels. The low molecular weight DNA is isolated by an established method (Wu W, Lee W L, Wu Y Y, Chen D, Liu T J, Jang A, Sharma P M; Wang P H., J. Biol. Chem 275(51):40113-9 (2000)), resolved with 1.2 % agarose gel containing ethidium bromide, and visualized under UV light. If laddering of DNA occurs, the DNA may be further end-labeled with ³²P, resolved with polyacrylamide gel electrophoresis, and exposed for analysis with densitometry if desired.

B. TUNEL Staining:

Paraffin sections of myocardial samples may be labeled with tdt-UTP nick end labeling (TUNEL) to detect DNA breakage in situ. To distinguish myocytes from non-myocytes, the sections are labeled with anti-tropomyosin antibodies and stained with anti-rabbit IgG-rhodamine. To verify that the green TUNEL staining is located in the nucleus, the nucleus is counterstained with DAPI. The apoptotic nuclei are stained green, non-apoptotic nuclei are blue, and cardiomyocytes are red under confocal fluorescence microscopy. Negative controls are obtained by omission of tdt enzyme during the reaction. The incidence of cardiomyocyte and non-myocyte apoptosis is calculated from 200 random microscopic fields in each section and recorded as per mm² of myocardium. The proportion of cardiomyocytes and non-myocytes undergoing apoptosis is estimated.

C. Caspase Activation:

The activities of caspase 3 may be determined with the CPP32 assay kit from Clontech (Palo Alto, Calif.). The cardiac tissue is solubilized, and 100 μg of lysate proteins are reacted with 50 μM DEVD-AFC at 37 C for 45 min. The samples are analyzed with a fluorescence measurement system at excitation of 425 nm and emission of 530 nm.

### Example 4

Treatment with a GLP-1 Molecule Increases Cardiac Contractility

A. Isolated Working Rat Heart Preparation:

Male Sprague-Dawley rats (250-300g) are anesthetized by using 5% isoflurane. The heart is rapidly excised, and placed in cold saline (4° C.). The heart is placed into a 5 temperature-controlled chamber (37° C.). After cannulating the aorta, constant pressure (80 mmHg) Langendorff (retrograde) perfusion is commenced. The perfusate contains a modified Krebs-Henseleit(KH) solution (NaCl 118 mM; KCl 4.7 mM;KH₂PO₄ 1.2 mM;MgSO₄ 1.2mM;Ca²⁺ 2.5 mM; Glucose 11 mM). The left atrium is cannulated through the pulmonary vein. After 15 min of retrograde perfusion, the heart is switched to the working heart mode and pre-ischemic function is evaluated at 11.5 mmHg (atrial filling pressure) with a 104 cm aortic column (afterload). During the working heart perfusion period, the heart is perfused with 1.2 mM palmitate +KH buffer with 100 μU/ml insulin.

To assess contractile function, a microtip pressure transducer catheter (Millar Instruments, Houston, Tex.) is inserted into the left ventricular cavity. Data are recorded using a PowerLab data acquisition system (ADI Instruments, Colorado Springs, Colo.).

In some studies, global ischemia is induced by simultaneously clamping both the aortic and atrial lines for 30 min. After ischemia, the heart is reperfused for 40 min. Measurements of cardiac outflow (CO) and aortic flow by transonic probes are performed at 10 min intervals throughout the experiment. Peak aortic systolic pressure, diastolic pressure, developed pressure (DP), and oxygen consumption (MVO₂) are measured. Cardiac work and efficiency are calculated. Cardiac work=DP×CO; Cardiac efficiency=Cardiac work/MVO₂.

[image]

All publications and patent applications cited herein are incorporated by reference to the same extent as if each individual publication or patent application was specifically and individually indicated to be incorporated by reference.

Although certain embodiments have been described in detail above, those having ordinary skill in the art will clearly understand that many modifications are possible in the embodiments without departing from the teachings thereof. All such modifications are intended to be encompassed within the claims of the invention.

What is claimed is:

1. A method for preventing or ameliorating apoptosis of cardiac myocytes in a subject in need thereof, said method comprising administering to said subject an amount of a GLP-1 molecule or agonist thereof effective to prevent apoptosis of cardiac myocytes.

2. The method according to claim 1, wherein said subject has congestive heart failure.

3. The method according to claim 1, wherein said subject has experienced or is experiencing myocardial infarction.

4. The method according to claim 1, wherein said subject has received a heart transplant.

5. The method according to claim 1, wherein said GLP-1 molecule or agonist thereof is acutely administered to said subject.

6. The method according to claim 1, wherein said GLP-1 molecule or agonist thereof is chronically administered to said subject.

7. The method according to claim 1, wherein said GLP-1 molecule is GLP-1.

8. The method according to claim 1, wherein said GLP-1 molecule is a GLP-1 analog with GLP-1 activity.

9. The method according to claim 1, wherein said GLP-1 molecule agonist is an exendin.

10. The method according to claim 9, wherein said exendin is an exendin-4 analog.

11. The method according to claim 9, wherein said exendin is exendin-4.

12. The method according to claim 1, wherein said GLP-1 molecule or agonist thereof is parenterally administered to said subject.

13. A method for the treatment or prevention of a condition associated with cardiac myocyte apoptosis in a subject in need thereof, said method comprising administering to said subject an amount of a GLP-1 molecule or agonist thereof effective to prevent cardiac myocyte apoptosis, wherein said condition associated with cardiac myocyte apoptosis is thereby improved.

14. The method according to claim 13, wherein said a GLP-1 molecule or agonist thereof is chronically administered to said subject.

15. The method according to claim 13, wherein said GLP-1 molecule or agonist thereof is acutely administered to said subject.

16. The method according to claim 13, wherein said GLP-1 molecule is GLP-1.

17. The method according to claim 13, wherein said GLP-1 molecule is a GLP-1 analog with GLP-1 activity.

18. The method according to claim 13, wherein said GLP-1 molecule agonist is an exendin.

19. The method according to claim 18, wherein said exendin is an exendin-4 analog.

20. The method according to claim 18, wherein said exendin is exendin-4.

21. The method according to claim 13, wherein said subject has diabetes.

22. The method according to claim 13, wherein said subject has hypertension.

23. The method according to claim 13, wherein said subject has congestive heart failure.

24. The method according to claim 13, wherein said subject has received a heart transplant.

25. The method according to claim 13, wherein said GLP-1 molecule or agonist thereof is parenterally administered to said subject.

26. A method for improving the efficiency of cardiac myocytes in a subject in need thereof, said method comprising administering to said subject an amount of a GLP-1 molecule or agonist thereof effective to improve the efficiency of cardiac myocytes.
